SEQUENCE LISTING (8 sequences)

SEQ ID NO: 1 - protein, 808 aa, Thermus sp. CCB_US3_UF1
MPLLPEPLSGLKELAYNLWWSWNPEAAELFQEIDPSLWKRFRGNPVKLLLEADPGRLEGLAATSYPARVGAVVEALRAYLREREEKQGPLVAYFSAEYGFHSSLPIYSGGLGVLAGDHVKAASDLGLNLVGVGIFYHEGYFHQRLSPEGVQVEVYETLHPEELPLYPVQDREGRPLRVGVEFPGRTLWLSAYRVQVGAVPVYLLTANLPENTPEDRAITARLYAPGLEMRIQQELVLGLGGVRLLRALGLAPEVFHMNEGHSAFLGLERVRELVAEGHPFPVALELARAGALFTTHTPVPAGHDAFPLELVERYLGGFWERMGTDRETFLSLGLEEKPWGKVFSMSNLALRTSAQANGVSRLHGEVSREMFHHLWPGFLREEVPIGHVTNGVHTWTFLHPRLRRHYAEVFGPEWRKRPEDPETWKVEALGEEFWQIHKDLRAELVREVRTRLYEQRRRNGESPSRLREAEKVLDPEALTIGFARRFATYKRAVLLFKDPERLRRLLHGHYPIQFVFAGKAHPKDEPGKAYLQELFAKIREYGLEDRMVVLEDYDMYLARVLVHGSDVWLNTPRRPMEASGTSGMKAALNGALNLSVLDGWWAEAYNGKNGFAIGDERVYESEEAQDMADAQALYDVLEFEVLPLFYAKGPEGYSSGWLSMVHESLRTVGPRYSAARMVGDLEIYRRGGAWAEAARAGQEALAAFHQALPALQGVTLRAQVPGDLTLNGVPMRVRAFLEGEVPEALRPFLEVQLVVRRSSGHLEVVPMRPGPDGYEVAYRPSRPGSYAYGVRLALRHPITGHVAWVRWA

SEQ ID NO: 2 - protein, 473 aa, Thermanaerothrix daxensis
MGHKIMFGTDGWRGVIAEDYTFDNVRRCAQGFAHYLKTKGYKDEWVVVGYDKRFHSENFAQAAAEVLCGNGFRVYLTDKATPTPVIAYAVVERKAIGAVNITASHNPPTDNGFKVRDASGGAIDPEGLKRIESAIPDEMSAVKRMPASEAEAQGRLVRFDPAPAYIEHLKSLIDLQPIRDAGLKIVVDAMWGNGAGWFPRLLAGGKTEVYEIHNTRNPIFPEMKRPEPIPPNIDVGLRTTVERRADVLVVTDGDADRVGIGDEHGRFVNQLQVYGLLAFYLLEVRGERGPIIKTLSTTSMLEKLGEIYGVPVYETGVGFKYVAPKFLETNALIGGEESGGYAFRGNVPERDGILAGLYFLDMMVRLNRKPSQLLELLFSKVGPHYYDRVDRQFTGDRKTREEMILNANPHTIGGLKVVGLNTLDGFKFLLEDGGWMLIRFSGTEPIIRVYCETTHPDRVQPILQDGLRIAGLA

SEQ ID NO: 3 - protein, 415 aa, Thermus thermophilus
MLRLDTRFLPGFPEALSRHGPLLEEARRRLLAKRGEPGSMLGWMDLPEDTETLREVRRYREANPWVEDFVLIGIGGSALGPKALEAAFNESGVRFHYLDHVEPEPILRLLRTLDPRKTLVNAVSKSGSTAETLAGLAVFLKWLKAHLGEDWRRHLVVTTDPKEGPLRAFAEREGLKAFAIPKEVGGRFSALSPVGLLPLAFAGADLDALLMGARKANETALAPLEESLPLKTALLLHLHRHLPVHVFMVYSERLSHLPSWFVQLHDESLGKVDRQGQRVGTTAVPALGPKDQHAQVQLFREGPLDKLLALVIPEAPLEDVEIPEVEGLEAASYLFGKTLFQLLKAEAEATYEALAEAGQRVYALFLPEVSPYAVGWLMQHLMWQTAFLGELWEVNAFDQPGVELGKVLTRKRLAG

SEQ ID NO: 4 - protein, 426 aa, Thermanaerothrix daxensis
MVTYLDFVVLSHRFRRPLGITSVCSAHPYVIEAALRNGMMTHTPVLIEATCNQVNQYGGYTGMTPADFVRYVENIAARVGSPRENLLLGGDHLGPLVWAHEPAESAMEKARALVKAYVEAGFRKIHLDCSMPCADDRDFSPKVIAERAAELAQVAESTCDVMGLPLPNYVIGTEVPPAGGAKAEAETLRVTRPEDAAETIALTRAAFFKRGLESAWERVVALVVQPGVEFGDHQIHVYRREEAQALSRFIESQPGLVYEAHSTDYQPRDALRALVEDHFAILKVGPALTFAFREAVFALASIEDWVCDSPSRILEVLETTMLANPVYWQKYYLGDERARRIARGYSFSDRIRYYWSAPAVEQAFERLRANLNRVSIPLVLLSQYLPDQYRKVRDGRLPNQFDALILDKIQAVLEDYNVACGVRIGE

SEQ ID NO: 5 - protein, 219 aa, Sphaerobacter thermophilus
MSQGVRGVVFDLDGLLVESEEYWEQARREFVSRYGGTWGDDAQQAVMGANTRQWSRYIREAFDIPLTEEEIAAAVIARMQELYHDHLPLLPGAIPAVRALADRYPLAVASSSPPVLIRFVLAEMGVAECFQSVTSSDEVAHGKPAPDVYHLACERLGVAPEQAVAFEDSTAGIAAALAAGLRVIAVPNRSYPPDPDVLRRADLTLPSLEEFDPAVLEQW

SEQ ID NO: 6 - protein, 511 aa, Anaerolinea thermophila
MSLFKRASGILLHPTSLPGPDGIGDLGPEAYRWVNFLAESGCSLWQILPLGPTGFGDSPYQCFSAFAGNPYLVSPALLLDEGLLTSEDLADRPEFPASRVDYGPVIQWKLTLLDRAYVRFKRSTSQKRKAAFEAFKEEQRAWLLDFSLFMAIKEAHGGASWDYWPEPLRKRDPEALNAFHRAHEVDVERHSFRQFLFFRQWQALRQYAHEKGVQIIGDVPIFVAYDSADVWSHPDLFYLDETGKPTVVAGVPPDYFSATGQLWGNPLYRWDYHRETGFAWWLERLKATFAMVDIVRLDHFRGFAGYWEVPYGMPTAEKGRWVPGPGIALFEAIRNALGGLPIIAEDLGEITPDVIELREQLGLPGMKIFQFAFASDADDPFLPHNYVQNCVAYTGTHDNDTAIGWYNSAPEKERDFVRRYLARSGEDIAWDMIRAVWSSVAMFAIAPLQDFLKLGPEARMNYPGRPAGNWGWRYEAFMLDDGLKNRIKEINYLYGRLPEHMKPPKVVKKWT

SEQ ID NO: 7 - protein, 225 aa, Thermoanaerobacterium thermosaccharolyticum
MKPMFAPSLMCANFLDLKNQIEILNERADIYHIDIMDGHYVKNFALSPYLMEQLKTIAKIPMDAHLMVENPADFLECIAKSGATYISPHAETINKDAFRIMRTIKALGCKTGIVLNPATPVEYIKYYIGMLDKITILTVDAGFAGQTFINEMLDKIAEIKSLRDQNGYSYLIEVDGSCNEKTFKQLAEAGTDVFVVGSSGLFNLDTDLKVAWDKMMDTFTRCTSN

SEQ ID NO: 8 - protein, 220 aa, Methanosarcina thermophila
MLKALIFDMDGVLVDSMPFHAAAWKKAFFEMGMEIQDSDIFAIEGSNPRNGLPLLIRKARKEPEAFDFEAITSIYRQEFKRVFEPKAFEGMKECLEVLKKRFLLSVVSGSDHVIVHSIINRLFPGIFDIVVTGDDIINSKPHPDPFLKAVELLNVRREECVVIENAILGVEAAKNARIYCIGVPTYVEPSHLDKADLVVEDHRQLMQHLLSLEPANGFRQ

(19) United States
(12) Patent Application Publication
(10) Pub. No.: US 2022/0259628 A1
(43) Pub. Date: Aug. 18, 2022

(54) IMMOBILIZED ENZYME COMPOSITIONS FOR THE PRODUCTION OF HEXOSES

(71) Applicant: BONUMOSE, INC., Charlottesville, VA (US)

(72) Inventors: Daniel Joseph WICHELECKI, Charlottesville, VA (US); Jonathan WAGNER, Waynesboro, VA (US)

(21) Appl. No.: 17/627,488

(22) PCT Filed: Jul. 17, 2020
(86) PCT No.: PCT/US20/42562

§ 371 Date: Jan. 14, 2022

Related U.S. Application Data
(60) Provisional application No. 62/875,321, filed on Jul. 17, 2019.
(60) Provisional application No. 62/924,323, filed on Oct. 22, 2019.

(51) Int. Cl. C12P 19/02; C12N 11/091; C12N 9/10; C12N 9/90; C12N 9/16

(52) U.S. Cl. CPC C12P 19/02; C12N 11/091; C12N 9/1051; C12Y 204/01001; C12N 9/90; C12Y 301/03; C12Y 503/01009; C12Y 501/03; C12N 9/107; C12Y 204/01018; C12N 9/16; C12Y 504/02002

(57) ABSTRACT

The invention relates to immobilized enzyme compositions for the preparation of a hexose. Hexoses include, for example, tagatose, psicose, fructose, allose, mannose, galactose, altrose, talose, sorbose, gulose, idose, and inositol. The invention also relates to an enzymatic process for preparing a hexose from a saccharide by contacting a starch derivative with an immobilized enzyme composition of the invention.

### CROSS REFERENCE TO RELATED APPLICATIONS

This application claims priority to U.S. Application No. 62/875,321, filed on Jul. 17, 2019, and U.S. Application No. 62/924,323, filed on Oct. 22, 2019, which are incorporated by reference herein in their entireties.

### FIELD OF THE INVENTION

The invention relates to preparation of hexose monosaccharides using immobilized enzyme compositions. More specifically, the invention relates to methods of preparing a D-hexose (or hexose) from saccharides (e.g., polysaccharides, oligosaccharides, disaccharides, sucrose, D-glucose, and D-fructose) including a step in which fructose 6-phosphate is converted to the hexose by one or more enzymatic steps catalyzed by immobilized enzymes.

### background

Hexoses are monosaccharides with six carbon atoms. Hexoses include, for example, tagatose, psicose, fructose, allose, mannose, galactose, altrose, talose, sorbose, gulose, idose, and inositol. Hexoses are used in numerous industries, clearly having a variety of applications in the pharmaceutical industry, biotechnology, and in the food and beverage industries. Hexoses may be prepared using enzymatic processes from saccharides, such as for example, monosaccharides, oligsaccharides, starch, starch derivatives, cellulose and the like. Solution-based enzymatic processes are described in published PCT applications WO 2018/169957, WO 2017/059278, and WO 2018/112139, which are incorporated herein by reference.

The commercial development of enzymatic processes is essential for increasing the number of industrial processes that utilize green chemistry and therefore decrease the impact of synthesis on the environment. Numerous enzymatic processes have been developed for commercial production (e.g., PCT applications WO 2018/169957, WO 2017/059278, and WO 2018/112139), but when these processes are for low-cost products (such as alternative sweeteners), hurdles to commercialization arise pertaining to the cost-in-use of enzyme. A common solution to this issue is the immobilization of enzymes to a carrier (e.g., WO 2016/160573). Immobilization allows for the enzyme to be reused between batches or to be used in a continuous process. The ability to reuse enzymes greatly reduces the cost-in-use of enzyme per kg of product and can be essential for commercial viability. Numerous methods for enzyme immobilization exist with no set rules for which method will be preferred by a specific process (Datta et al., Enzyme immobilization: an overview on techniques and support materials. 3 Biotech (2013), 3:1-9). Therefore, unique solutions must be developed for each process.

The enzymes in commercial processes can be adsorbed on insoluble organic or inorganic supports commonly used to improve functionality, as known in the art. These include polymeric supports such as agarose, methacrylate, polystyrene, phenol formaldehyde, or dextran, as well as inorganic supports such as glass, metal, or carbon-based materials. These materials are often produced with large surface-to-volume ratios and specialized surfaces that promote attachment and activity of immobilized enzymes. The enzymes might be affixed to these solid supports through covalent, ionic, or hydrophobic interactions. The enzymes could also be affixed through genetically engineered interactions such as covalent fusion to another protein or peptide sequence with affinity to the solid support, most often a polyhistidine sequence. The enzymes might be affixed either directly to the surface or surface coating, or they might be affixed to other proteins already present on the surface or surface coating. The enzymes can be immobilized all on one carrier, on individual carriers, or a combination of the two (e.g., two enzyme per carrier then mix those carriers). These variations can be mixed evenly or in defined layers to optimize turnover in a continuous reactor. These enzymes may be mixed evenly or in defined layers or zones to optimize turnover. For example, the beginning of the reactor may have a layer of aGP to ensure a high initial G1P increase. Enzymes may be immobilized all on one carrier bead, each on an individual carrier bead, or in groups of enzymes on a carrier bead. Similarly the enzymes may be immobilized on a specific carrier or multiple carriers within one process using one or more immobilization methodologies.

There is a need to have improved processes for the production of hexoses that allows for scalability and reuseable enzyme compositions while achieving high yields of the desired hexose.

### SUMMARY OF THE INVENTION

The invention relates to immobilized enzyme compositions for the preparation of a hexose. Hexoses include, for example, tagatose, psicose, fructose, allose, mannose, galactose, altrose, talose, sorbose, gulose, idose, and inositol. An immobilized enzyme composition of the invention comprises, consists essentially of, or consists of at least two, at least three, at least four, at least five, at least six carriers, at least seven, or at least eight of the following enzymes immobilized to at least one carrier or a mixture of carriers:

a) α glucan phosphorylase (αGP), phosphoglucomutase (PGM), and optionally 1,4-glucan transferase (4-GT); and

b) an enzyme from within a combination of enzymes selected from:
(i) phosphoglucoisomerase (PGI), fructose-6-phosphate epimerase (F6PE), and tagatose-6-phosphate phosphatase (T6PP) to prepare tagatose;
(ii) phosphoglucoisomerase (PGI), piscose-6-phosphate epimerase (P6PE), and picose-6-phosphate phosphatase (P6PP) to prepare allulose;
(iii) phosphoglucoisomerase (PGI), P6PE, allose-6-phosphate isomerase (A6PI), and allose-6-phosphate phosphatase (A6PP) to prepare allose;
(iv) phosphoglucoisomerase (PGI), mannose-6-phosphate isomerase (M6PI) or phosphoglucose/phosphomannose isomerase (PGPMI), and mannose 6-phosphate phosphatase (M6PP) to prepare mannose;
(v) phosphoglucoisomerase (PGI), F6PE, galactose 6-phosphate isomerase (Gal6PI), and galactose 6-phosphate phosphatase (Gal6PP) to prepare galactose;
(vi) PGI and fructose 6-phosphate phosphatase (F6PP) to prepare fructose;
(vii) PGI, P6PE, altrose 6-phosphate isomerase (Alt6PI), and altrose 6-phosphate phosphatase (Alt6PP) to prepare altrose;
(viii) PGI, F6PE, talose 6-phosphate isomerase (Tal6PI), and talose 6-phosphate phosphatase (Tal6PP) to prepare talose;
(ix) PGI, F6PE, sorbose 6-phosphate epimerase (S6PE), and sorbose 6-phosphate phosphatase (S6PP) to prepare sorbose;
(x) PGI, F6PE, S6PE, gulose 6-phosphate isomerase (Gul6PI), and gulose 6-phosphate phosphatase (Gul6PP) to prepare gulose;
(xi) PGI, F6PE, S6PE, idose 6-phosphate isomerase (I6PI), and idose 6-phosphate phosphatase (I6PP) to prepare idose; and
(xii) inositol 3-phosphate synthase (IPS) and inositol monophosphatase (IMP) to prepare inositol.
   In an immobilized enzyme composition of the invention the weight of each enzyme relative to the total weight of the enzymes (w/w) % ranges from 0.1% to 40%.

The invention also relates to an enzymatic process for preparing a hexose from a saccharide by contacting a starch derivative with an immobilized enzyme composition of the invention under suitable reaction conditions convert the starch derivative to the hexose.

### BRIEF DESCRIPTION OF THE FIGURES

FIG. 1A is a graph showing the amounts of enzyme in an immobilized enzyme composition that is optimized for tagatose production.

FIG. 1B is a graph showing the amounts of enzyme in an immobilized enzyme composition in which the amounts are normalized based on the observed reaction rate of each enzyme relative to T6PP activity in order to have equal units of activity within the cascade.

FIG. 1C is a graph showing the amounts of enzyme in an immobilized enzyme composition in which the amounts were in w/w ratios of 1:1:1:1:1:1.

FIG. 2 is a graph showing the relationship of enzyme loading (w/w % of total enzyme weight/carrier weight) on the activity of an immobilized enzyme composition that is optimized for tagatose production on DUOLITE™ A568. Results shown are based on relative enzymatic cascade rates with respect to a 5% loaded carrier.

### DETAILED DESCRIPTION

The following description discloses the invention according to embodiments related to making and using enzymes that are immobilized to a carrier (“immobilized enzyme compositions”) in processes for converting starches and starch derivatives and saccharides to hexose monosaccharides, including, for example, tagatose, psicose, fructose, allose, mannose, galactose, altrose, talose, sorbose, gulose, idose, and inositol. These processes can generally be described as enzymatic reactions that create a phosphorylated intermediate from starch, a starch derivative, or a saccharide using free phosphate (no ATP). The free phosphate is released in a highly energetically favorable final step to produce a hexose-of-interest (e.g., tagatose, psicose, fructose, allose, mannose, galactose, altrose, talose, sorbose, gulose, idose, or inositol). The phosphate is then recycled to convert additional starch, starch derivative, or a saccharide into a phosphorylated intermediate so that the process can repeat. This allows for non-stoichiometric amounts of phosphate to be utilized which lowers the cost of phosphate use in the process and limits phosphate levels in waste.

Embodiments of the invention include compositions of at least two, at least three, at least four, at least five, at least six carriers, at least seven, or at least eight enzymes, that are immoblized on a carrier, and, which catalyze, respectively, reactions in an enzymatic process for converting a starch, starch derivative, and/or a saccharide to a hexose. An immobilized enzyme composition of the invention provides numerous advantages over their use in free solution, including: longer duration of activity (due to protection of structural features of the protein), multiple cycle reuse, and elimination of the need to remove the enzyme downstream. In addition, immobilizing enzymes on solid surfaces can work in either a stirred tank reactor, a packed bed reactor, or a rotating bed reactor; allowing flexibility in scale-up.

The enzymes contained in an immobilzed enzyme composition of the invention catalyze at least two, at least three, at least four, at least five, at least six carriers, at least seven, or at least eight, reactions involved in the stepwise conversion of a starch, starch derivative, or saccharides to a hexose. The following patent publications, which are all disclosed herein in their entireties, disclose enzymatic processes, (i.e., enzyme reaction cascades) for producing hexoses in solution: published PCT applications WO 2018/169957, WO 2017/059278, and WO 2018/112139. Immobilized enzyme compositions of the invention, can include, but are not limited to, any of the enzymes and enzyme combinations disclosed in these references.

Some immobilized enzyme compositions of the invention contain a combination of enzymes that catalyze reactions, which are common among processes for producing different hexoses, such as reaction steps leading to conversion of glucose 6-phosphate (G6P) to fructose 6-phosphate (F6P). The enzymes that catalyze these common reaction steps may be referred to as “core enzymes”. Accordingly, in some immobilized enzyme compositions of the invention, an immobilized enzyme composition at least contains the core enzymes, α glucan phosphorylase (αGP) to convert a saccharide to glucose 1-phosphate (G1P) and phosphoglucomutase (PGM) to convert G1P to glucose 6-phosphate (G6P). Enzymes in an immobilized enzyme composition of the invention, which catalyze additional reaction steps to convert G6P to various hexose products can be coimmobilized with core enzymes, or contained in separate immobilized enzyme compositions.

Typically, core enzymes are combined in an immobilized composition with one or more of enzymes used in the production of tagatose, psicose, fructose, allose, mannose, galactose, altrose, talose, sorbose, gulose, idose, or inositol. Accordingly, in addition to αGP and PGM, some immobilized enzyme compositions according to the invention also contain: (i) phosphoglucoisomerase (PGI), fructose-6-phosphate epimerase (F6PE), and tagatose-6-phosphate phosphatase (T6PP) to prepare tagatose; (ii) PGI, piscose-6-phosphate epimerase (P6PE), and picose-6-phosphate phosphatase (P6PP) to prepare allulose; (iii) PGI, P6PE, allose-6-phosphate isomerase (A6PI), and allose-6-phosphate phosphatase (A6PP) to prepare allose; (iv) PGI, mannose-6-phosphate isomerase (M6PI) or phosphoglucose/phosphomannose isomerase (PGPMI), and mannose 6-phosphate phosphatase (M6PP) to prepare mannose; (v) PGI, F6PE, galactose 6-phosphate isomerase (Gal6PI), and galactose 6-phosphate phosphatase (Gal6PP) to prepare galactose; (vi) PGI and fructose 6-phosphate phosphatase (F6PP) to prepare fructose; (vii) PGI, P6PE, altrose 6-phosphate isomerase (Alt6PI), and altrose 6-phosphate phosphatase (Alt6PP) to prepare altrose; (viii) PGI, F6PE, talose 6-phosphate isomerase (Tal6PI), and talose 6-phosphate phosphatase (Tal6PP) to prepare talose; (ix) PGI, F6PE, sorbose 6-phosphate epimerase (S6PE), and sorbose 6-phosphate phosphatase (S6PP) to prepare talose; (x) PGI, F6PE, S6PE, gulose 6-phosphate isomerase (Gul6PI), and gulose 6-phosphate phosphatase (Gul6PP) to prepare gulose; (xi) PGI, F6PE, S6PE, idose 6-phosphate isomerase (I6PI), and idose 6-phosphate phosphatase (I6PP) to prepare idose; and (xii) inositol 3-phosphate synthase (IPS) and inositol monophosphatase (IMP) to prepare inositol. Each combination of core enzymes and an enzyme composition (i)-(xii) are separate embodiments of the invention.

The immobilized enzyme compositions, above, can also optionally contain 4-glucan transferase (4GT). 4GT can be used to increase hexose yields by recycling the degradation products glucose, maltose, and maltotriose into longer maltooligosaccharides; which can be phosphorolytically cleaved by αGP to yield G1P.

The relative weight ratios between enzymes in an immobilized enzyme composition of the invention may range from 1:1000 to 1000:1, from 1:100 to 100:1 or from 1:50 to 50:1, when comparing any two enzymes in the immobilized composition. The enzyme ratios, including other optional enzymes discussed below, can be varied to increase the efficiency of hexose production. For example, a particular enzyme may be present in an amount about 2×, 3×, 4×, 5×, 10× etc. relative to the amount of another enzyme.

Relative weight/weight ratios among the enzymes in an immobilized enzyme composition of the invention can be optimized to increase process performance and/or hexose yield. In that regard, the weight of each enzyme in an immobilized enzyme composition, relative to the total weight of the enzymes (w/w) % ranges from 0.1% to 70%. For example, some immobilized enzyme compositions of the invention contain 10-30% (αGP); 10-30% (PGM); and when present 0.1-10% (PGI) and 0.1-10% (4GT), when present. For example, an immobillized enzyme composition of the invention that can be used for the production of tagatose, the weight of each enzyme relative to the total weight of the enzymes (w/w) % is: 10-30% (αGP); 0-10% (4GT); 10-30% (PGM); 0.1-10% (PGI); 15-35% (F6PE); and T6PP (25-45%), wherein the total weight of enzymes in the composition is 100 w/w % relative to the total weight of the enzymes. Some immobilized enzyme compositions of the invention for use in tagatose production contain: 19% αGP; 3% 4GT; 17% PGM; 3% PGI; 23% F6PE; and T6PP 35%, wherein the % weight of each enzyme is relative to the total weight of the enzymes in the immobilized enzyme composition of the invention, and the total weight of enzymes in the composition is 100 w/w % relative to the total weight of the enzymes. In other examples, an immobillized enzyme composition of the invention for used for the production of allulose, in which the weight of each enzyme relative to the total weight of the enzymes (w/w) % is: 10-30% (αGP); 0-10% (4GT); 10-30% (PGM); 0.1-10% (PGI); 0.1-10% (P6PE); and (45-65%) P6PP, wherein the total weight of enzymes in the composition total 100 w/w % relative to the total weight of the enzymes. Some immobilized enzyme compositions of the invention for use in allulose production contain: 20% αGP; 3% 4GT; 16.5% PGM; 3% PGI; 3% P6PE; and P6PP 54%, wherein the % weight of each enzyme is relative to the total weight of the enzymes in the immobilized enzyme composition of the invention, and the total weight of enzymes in the composition is 100 w/w % relative to the total weight of the enzymes.

While enzymes contained in immobilized compositions of the invention are generally referred to based on the reactions they catalyze, (i.e., by specificity and function), enzymes are also commonly identified by amino acid sequence, (e.g., a SEQ. ID. NO.; a database indentification number, such as a UniProt ID), amino acid sequence identity/similarity to an enzyme of known function, nucleotide sequence, or nucleotide sequence identity/similarity to an enzyme of known function. Enzymes known in the art and used to prepare hexoses may be used in an immobilized enzyme composition of the invention, including immobilized enzyme compositions that can be used to produce , tagatose, psicose, fructose, allose, mannose, galactose, altrose, talose, sorbose, gulose, idose, and inositol. Exemplary enzymes known in the art may be used in a immoblized enzyme composition of the invention are identified below with a relevant patent document. The disclosure of the enzymes in the listed patents is specifically incorporated here by reference.

| αGP | PGM | PGI |
|---|---|---|
| WO2018/129275A1 | WO2018/129275A1 | WO2018/129275A1 |
| WO2017059278 | WO2017059278 | WO2017059278 |
| WO2018/112139 | WO2018/112139 | WO2018/112139 |
| PCT/US2019/056978 | PCT/US2019/056978 | PCT/US2019/056978 |
| PCT/US2019/058483 | PCT/US2019/058483 | PCT/US2019/058483 |
| US20190194696A1 | US20190194696A1 | US20190194696A1 |
| CN106811493A | | |
| F6PE | T6PP | 4GT |
| WO2015/016544 | WO2018004310A1 | WO2017059278 |
| WO2017059278 | WO2017059278 | WO2018/112139 |
| PCT/US2019/056978 | PCT/US2019/056978 | PCT/US2019/056978 |
| PCT/US2019/058483 | PCT/US2019/058483 | PCT/US2019/058483 |
| | | US20190194696A1 |
| | | CN106811493A |
| P6PE | P6PP | |
| WO2018004308A2 | WO2018/112139 | |
| WO2018/129275A1 | WO2018/129275A1 | |
| WO2018/112139 | | |

Accordingly, in certain immobilized enzyme compositions, Table 1 provides a UniProt ID and SEQ. ID No. to identify the amino acid sequences for the following non-limiting examples of enzymes that can be included in immobilized enzyme compositions of the invention: αGP, PGM, PGI, F6PE, T6PP, 4-GT, P6PE, and P6PP.

TABLE 1

| Enzyme | UniProt ID (SEQ. ID. NO.) |
|---|---|
| α glucan phosphorylase (αGP) | G8NCC0 (SEQ. ID. NO. 1) |
| Phosphoglucomutase (PGM) | A0A0P6YKY9 (SEQ. ID. NO. 2) |
| Phosphoglucoisomerase (PGI) | Q5SLL6 (SEQ. ID. NO. 3) |
| fructose-6-phosphate epimerase | A0A0P6XN50 (SEQ. ID. NO. 4) |
| (F6PE) | |
| tagatose-6-phosphate phosphatase | D1C7G9 (SEQ. ID. NO. 5) |
| (T6PP) | |
| 1,4-glucan transferase (4-GT) | E8MXP8 (SEQ. ID. NO. 6) |
| psicose 6-phosphate 3-epimerase | A0A223HZ17 (SEQ. ID. NO. 7) |
| (P6PE) | |
| psicose 6-phosphate phosphatase | A0A0E3NCH4 (SEQ. ID. NO. 8) |
| (P6PP) | |

The amino acid sequences of enzymes contained in immobilized enzyme compositions of the invention also include enzymes, which may have been modified for any reason, such as, for example, to improve activity, stability (i.e., half-life), or yield. Such modified enzymes include, for example, fragments of enzymes, amino acid substitutions, and chimeric proteins. Modified enzymes includes variants of any enzyme disclosed herein. Variants may contain amino acid substitutions at one or more amino acid residues. A variant includes no more than 15, no more than 12, no more than 10, no more than 9, no more than 8, no more than 7, no more than 6, no more than 5, no more than 4, no more than 3, no more than 2 or no more than 1 conservative amino acid substitution relative to a naturally occurring enzyme and/or no more than 5, no more than 4, no more than 3, or no more than 2 non-conservative amino acid substitutions, or no more than 1 non-conservative amino acid substitution, relative to a naturally occurring enzymes. A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). Variant enzymes of the invention may include amino acid substitutions with amino acid analogs as well as amino acids, as described herein.

An enzyme contained in an enzyme composition of the invention can also be an enzyme, which: i) shares at least 35% sequence identity with the amino acid sequence of an enzyme disclosed herein; and ii) can catalyze the same reaction of that particular disclosed enzyme with the necessary specificity for the process. Accordingly, an enzyme in a composition of the invention includes enzymes with amino acid sequences that are at least 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% 90% 91%, 92%, 93%, 94%, 95%, 96%, 97%, 98%, or 99% sequence identity with the amino acid sequence of an enzyme disclosed herein. As used herein, the term “sequence identity” refers to the similarity between two, or more, amino acid or nucleic acid sequences. Sequence identity is typically measured in terms of percentage identity (or similarity or homology) between amino acid sequences; the higher the percentage, the more similar to each other are the compared sequences.

As described above, enzyme compositions of the invention are immobilized to at least one carrier. A carrier may also be generally referred to herein as a “carrier material”, “carrier resin”, or a “carrier bead”. In an immobilized enzyme composition of the invention, multiple different carriers may be used to immobilize one or more enzymes. While carriers in immobilized enzyme compositions of the invention are not necessarily limited to a particular material, in some immobilized enzyme compositions of the invention, the carrier is a weak base ion exchange resin, which may, optionally, be composed of phenol formaldehyde, (i.e., a phenol formaldehyde polycondensate). In yet other immobilized enzyme compositions of the invention, the carrier material is based on controlled pore glass (CPG) particles or hybrid CPG particles (WO2015115993A1). The carriers in some immobilized enzyme compositions of the invention are functionalized by the inclusion of a tertiary amine group, while in others, a secondary amine group provides functionality. In some immobilized enzyme compositions of the invention, a carrier resin is functionalized with groups used to chelate a metal, such as, for example, iron or zinc. The chelated metal groups allow high affinity binding of molecules via appropriate binding group, such as, for example, a Histidine (His)-tag. Examples of such functional groups are found in WO2015115993A1 and Cassimjee et al. A general protein purification and immobilization method on controlled porosity glass: biocatalytic applications. Chem. Commun., 2014, 50, 9134; including but not limiting to 2,4-dihydroxybenzyl residues.

The carriers in some immobilized enzyme compositions of the invention possess two or more of the various carrier features that are listed above. For example, in some immobilized enzyme compositions of the invention, the enzyme compositions of any of the immobilized enzyme compositions described, herein, for use in processes for converting starches and starch derivatives and saccharides to hexose monosaccharides, including processes for producing tagatose, psicose, fructose, allose, mannose, galactose, altrose, talose, sorbose, gulose, idose, or inositol, are immobilized to a weak base anion exchange resin; which may, or may not be composed of phenol formaldehyde polycondensate; and which may, or may not be functionalized by a tertiary amine group. A carrier resin with the foregoing features is sold commercially as DUOLITE™ A568. In other immobilized enzyme compositions of the invention, the enzyme compositions of any of the immobilized enzyme compositions described, herein, for use in processes converting starches and starch derivatives and saccharides to hexose monosaccharides, including processes for producing tagatose, psicose, fructose, allose, mannose, galactose, altrose, talose, sorbose, gulose, idose, or inositol, are immobilized to a weak base anion exchange resin; which may, or may not be composed of phenol formaldehyde polycondensate; and which may, or may not be functionalized by a secondary amine group. A carrier resin with the foregoing features is sold commercially as DUOLITE™ PWA7. In yet other immobilized enzyme compositions of the invention, the enzyme compositions of any of the immobilized enzyme compositions described, herein, for use in processes converting starches and starch derivatives and saccharides to hexose monosaccharides, including processes for producing tagatose, psicose, fructose, allose, mannose, galactose, altrose, talose, sorbose, gulose, idose, or inositol, are immobilized to a His-tag affinity resin; which may, or may not be composed of a CPG or hybrid CPG particle material carrier; and which may, or may not chelate iron or zinc. A carrier resin with the foregoing features is sold commercially as EziG™ Opal.

In general, the total weight of the enzymes in an immobilized enzyme composition of the invention, relative to the weight of the carrier (w/w) %, range from 2.5%-12.5%. Therefore, the total weight of the enzyme compositions of any of the immobilized enzyme compositions described, herein, for use in processes converting starches and starch derivatives and saccharides to hexose monosaccharides, including processes for producing tagatose, psicose, fructose, allose, mannose, galactose, altrose, talose, sorbose, gulose, idose, or inositol, relative to the weight of the carrier (w/w) %, can be about 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, or any w/w % therein. For example, the total weight of the enzymes, (αGP, PGM, PGI, F6PE, and T6PP) or (αGP, 4GT, PGM, PGI, F6PE, and T6PP) in some immobilized enzyme compositions used to produce tagatose is 5%, while in the total weight of the enzymes (αGP, PGM, PGI, P6PE, and P6PP) or (αGP, 4GT, PGM, PGI, P6PE, and P6PP) is 6.5%.

The invention also relates to an enzymatic process for preparing a hexose from a saccharide comprising the step of contacting a starch derivative with an immobilized enzyme composition of the invention under suitable reaction conditions to convert the starch derivative to the hexose. In an enzymatic processes of the invention for preparing a hexose, the at least two, at least three, at least four, at least five, at least six carriers, at least seven, or at least eight enzymes of a process may be immobilized on the same carrier, or immobilized on multiple carriers. For example, in an enzymatic process of the invention the enzymes may be immobilized on the same carrier, or the immobilized enzymes may be distributed among at least two, at least three, at least four, at least five, at least six carriers, at least seven, or at least eight carriers, which may be the same type of carrier, or any combination of different carriers and immobilization methodolgies, including weak base anion exchange resin carriers, phenol formaldehyde polycondensate carriers, carriers that a comprise tertiary amine functional group, (e.g., DUOLITE™ A568), carriers that comprise a secondary amine functional groups, (e.g., DUOLITE™ PWA7), carriers that comprise a His-tag affinity resin, carriers that comprise controlled pore glass (CPG) particles, and carriers functionalized by a chelated metal, including carriers in which the chelated metal is iron or zinc (e.g., EziG™ Opal).

Methods for immobilizing enzymes to any of the carriers described here, including appropriate buffer and reaction conditions for binding enzymes to carrier resins, are known in the art. See, for example, WO 2016/160573, which is incorporated here in its entirety

Processes for converting starches and starch derivatives and saccharides to hexoses using immobilized enzyme compositions of the invention can be performed under the same temperature, buffer, and reaction time paramenters used for the production of hexoses using non-immobilized enzymes in solution. For example, immobilized enzyme compositions of the invention can be used to produce tagatose, psicose, fructose, allose, mannose, galactose, altrose, talose, sorbose, gulose, idose, or inositol from starches and starch derivatives and saccharides under the reaction conditions described in published PCT applications WO 2018/169957, WO 2017/059278, and WO 2018/112139. The multiple catalyzation reaction steps in a process of producing a hexose using an immobilized enzyme composition of the invention can be conducted in a single bioreactor, or in a plurality of bioreactors that are arranged in series, or a reaction vessel. Alternatively, the steps can also be conducted in a plurality of bioreactors, or reaction vessels, that are arranged in series or parallel. All aforementioned processes can be run in batch mode or continuous mode. A “one-pot” process in a single bioreactor is preferred.

The steps in an ezymatic processes of the invention can be conducted at a temperature ranging from about 35° C. to about 90° C., about 40° C. to about 70° C., about 50° C. to about 60° C. or about 55° C. and at a pH ranging from about 5.0 to about 8.0, about 6.5 to about 7.5 or about 7.0 to about 7.5. They may be conducted for about 0.5 hours to about 48 hours, about 4 hours to 24 hours or about 8 hours to 12 hours. The enzymatic process steps of the inventions may be conducted ATP-free and/or NAD(P)(H)-free. The steps can be carried out at a phosphate concentration ranging from about 0.1 mM to about 150 mM. The phosphate used in the phosphorylation and dephosphorylation steps of the processes according to the inventions can be recycled within the enzymatic cascade reaction. The processes of the invention can be carried out in a packed column or in a slurry.

For example, reaction phosphate concentrations in each of the processes can range from about 0.1 mM to about 300 mM, from about 0 mM to about 150 mM, from about 1 mM to about 50 mM, preferably from about 5 mM to about 50 mM, or more preferably from about 10 mM to about 50 mM. For instance, the reaction phosphate concentration in each of the processes can be about 0.1 mM, about 0.5 mM, about 1 mM, about 1.5 mM, about 2 mM, about 2.5 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, or about 55 mM.

Low phosphate concentration results in decreased production costs due to low total phosphate and thus lowered cost of phosphate removal. It also prevents inhibition of process enzymes by high concentrations of free phosphate and decreases the potential for phosphate pollution.

Furthermore, each of the processes disclosed herein can be conducted without added ATP as a source of phosphate, i.e., ATP-free. Each of the processes can also be conducted without having to add NAD(P)(H), i.e., NAD(P)(H)-free. Other advantages also include the fact that at least one step of the disclosed processes for making a hexose involves a highly energetically favorable chemical reaction which is essential for high yields. A highly energetically favorable chemical reaction for a process of the invention has an equilibrium constant (K_{eq}) of at least 2, at least 3, or at least 4.

As a first step in a process of the invention, the starch derivatives can be prepared by enzymatic hydrolysis of starch or by acid hydrolysis of starch. See, for example, WO 2017/059278. For example, the enzymatic hydrolysis of starch can be catalyzed or enhanced by isoamylase (IA, EC. 3.2.1.68), which hydrolyzes α-1,6-glucosidic bonds; pullulanase (PA, EC. 3.2.1.41), which hydrolyzes α-1,6-glucosidic bonds; 4-α-glucanotransferase (4GT, EC. 2.4.1.25), which catalyzes the transglycosylation of short maltooligosaccharides, yielding longer maltooligosaccharides; or alpha-amylase (EC 3.2.1.1), which cleaves α-1,4-glucosidic bonds. Furthermore, derivatives of cellulose can be prepared by enzymatic hydrolysis of cellulose catalyzed by cellulase mixtures, by acids, or by pretreatment of biomass.

### EXAMPLES

Example 1. Evaluation of enzyme carriers. Enzyme carriers were evaluated for relative reaction rates of enzyme compositions for producing tagatose, as well as for their effect on enzyme stability. With those objectives in mind, the following enzyme carrier materials were evaluated: Four branded phenol-formaldehyde matrix resins (DUOLITE™ A568, DUOLITE™ A561, DUOLITE™ PWA7, AmberLite™ FPA54); Two branded polystyrene resins (Lifetech™ ECR1640 and LifeTech™ ECR1504); Two branded polymethylacrylate resins (Lifetech™ ECR8309M and Chromolite™ D6154); and a branded controlled pore glass resin (EziG Opal™).

DUOLITE™ A568 (DuPont) Ion Exchange Resin is a highly porous, granular, weak base anion exchange resin based on crosslinked phenol-formaldehyde polycondensate. Its hydrophilicity and controlled pore size distribution make it the most suitable resin to be used as an enzyme carrier in many bioprocessing applications. The ionic strength, pore volume, pore size, and particle size of DUOLITE™ A568 are designed for optimum immobilization of enzymes used in the starch and fat (and other) industries.

DUOLITE™ A561 Ion Exchange Resin is a weak basic anion exchanger made from phenol formaldehyde with a tertiary amine functional group, and its broad specifications are similar to DUOLITE™ A568 resin, but DUOLITE™ A561 has a different bead morphology.

DUOLITE™ PWA7 is a weakly basic anion exchanger with amine functionality, and salt form.

AMBERLITE™ FPA54 Ion Exchange Resin is a highly porous, weak base, anion exchange resin, based on a crosslinked phenol-formaldehyde matrix. The low-swelling characteristics of AMBERLITE™ FPA54 give it excellent osmotic and physical stability resulting in less product loss and longer product life than conventional styrenic resins in food processing and bioprocessing applications. The hydrophilic phenolic, porous matrix of AMBERLITET™ FPA54 permits the reversible adsorption of high molecular weight, organic, color bodies frequently found in solutions of natural product and fermentation products. AMBERLITE™ FPA54 exhibits a high selectivity for sulfates and phosphates and, therefore, makes it ideal for the treatment of both citric and lactic acids derived from fermentation where it has a long history of use, particularly due to its excellent osmotic stability.

Lifetech™ ECR1640 is a copolymer of divinylbenzene (DVB) and styerene functionalised with quaternary amines. It is used for enzyme immobilization by ionic interaction of the ionizable surface aminoacids (Lys, Arg, His, Asp, Glu) with the tertiary amines on the polymer. It is particularly suitable for immobilization of enzymes with pls in the range 3-5 like many glycosidases. Lifetech™ ECR1640 main features are the possibility to regenerate the resin, pH adjustment before immobilization and large particle size for column applications. DVB/styerene with quaternary amines, 300-1200 micron, pH stability 1-14, supplied wet (66-72% water),capacity 0.85 eq/l Cl-form.

Lifetech™ ECR1504 is a copolymer of divinylbenzene (DVB) and styerene functionalised with tertiary amines. It is used for enzyme immobilization by ionic interaction of the ionizable surface aminoacids (Lys, Arg, His, Asp, Glu) with the tertiary amines on the polymer. It is particularly suitable for immobilization of enzymes with pl in the range 3-5 like many glycosidaseses. Lifetech™ ECR1504 main features are possibility to regenerate the resin, pH adjustment before immobilization and large particle size for column applications. DVB/styerene with tertiary amines, 300-1200 micron, pH stability 1-14, supplied wet (53-62% water), capacity 1.3 eq/l free base.

Lifetech™ ECR8309M is a hydrophilic, high porosity, methacrylate polymer functionalized with amino groups on a short spacer (C2).

Chromolite™ D6154 is macroporous polymethacrylate is a material, functionalized for affinity binding specific for poly His-tags. The functional group is iminodiacetic, Na form

EziG™ Opal is made from controlled pore glass (CPG) particles, and has a hydrophilic surface. The material has a narrow pore size distribution, produced with a pore diameter of ˜500 Å as standard. A mass loading of 15-60% active enzyme is expected. Other variations of EziG™ exist and were tested (Amber and Coral), but they performed suboptimally compared to Opal.

An enzyme composition containing the following weight/weight (w/w) percentages of each of the following enzymes, respectively, relative to to the total composition enzyme weight: 19% α-glucan phosphorylase (αGP, UNIPROT ID G8NCCO, SEQ. ID. NO. 1); 17% Phosphoglucomutase (PGM, UNIPROT ID A0A0P6YKY9, SEQ. ID. NO. 2); 3% phosphoglucoisomerase (PGI, UNIPROT ID Q5SLL6, SEQ. ID. NO. 3); 23% fructose-6-phosphate epimerase (F6PE, UNIPROT ID A0A0P6XN50, SEQ. ID. NO. 4); 35% tagatose-6-phosphate phosphatase (T6PP, UNIPROT ID D1C7G9, SEQ. ID. NO. 5); and 3% 1,4-glucan transferase (4-GT, UNIPROT ID E8MXP8, SEQ. ID. NO. 6), was immobilized on to each of the carriers described above. All immobilized enzyme preparations were 5% total enzyme weight/carrier. See Table 2. The foregoing enzyme ratios were previously optimized for tagatose production using the immobilization methodology described below.

TABLE 2

| | | % of enzyme |
|---|---|---|
| Enzyme | Uniprot ID | composition |
| α glucan phosphorylase (αGP) | G8NCC0 | 19% |
| | (SEQ. ID. NO. 1) | |
| Phosphoglucomutase (PGM) | A0A0P6YKY9 | 17% |
| | (SEQ. ID. NO. 2) | |
| Phosphoglucoisomerase (PGI) | Q5SLL6 | 3% |
| | (SEQ. ID. NO. 3) | |
| fructose-6-phosphate epimerase | A0A0P6XN50 | 23% |
| (F6PE) | (SEQ. ID. NO. 4) | |
| tagatose-6-phosphate phosphatase | D1C7G9 | 35% |
| (T6PP) | (SEQ. ID. NO. 5) | |
| 1,4-glucan transferase (4-GT) | E8MXP8 | 3% |
| | (SEQ. ID. NO. 6) | |

Prior to adhering the enzymes in the composition to each carrier, the carrier was equilibrated with two equivalent volumes of water, and then three equivalent volumes of immobilization buffer pH 7.2 (5 mM Na₂PO₄, 5 mM MgSO₄, 0.25 mM MnCl₂). The enzymes were suspended in the immobilization buffer to form the enzyme composition (preferably between 5 and 10 g/L enzyme) which was then added to the carrier to make a slurry. During room temperature incubation on an orbital shaker at 800 rpm, the absorbance of the enzyme composition and carrier slurry was measured at 280 nm to track the adsorbtion of the enzymes to the carrier until >95% of the soluble enzymes were no longer suspended in solution, which was around 6 hrs for a 5% (w/w, enzymes/carrier) loaded sample. The supernatant was removed and immobilized carrier washed with reaction buffer pH 7.2 (25 mM Na₂PO₄, 4 mM Na₂SO₃, 2.5 mM MgSO₄, 0.25 mM MnCl₂) to remove any remaining soluble enzymes. Each slurry sample was mixed with an equal volume of a 2× concentrated feed solution (320 g/L maltodextrin dextrose equivalent 5 (DE 5) pH 7.2, 25 mM Na₂PO₄, 4 mM Na₂SO₃, 2.5 mM MgSO₄, 0.25 mM MnCl₂) to achieve a final maltodextrin substrate concentration of 160 g/L in reaction conditions. The maltodextrin—carrier mixture was shaken overnight in a 2.0 mL microfuge tube in an Eppendorf Thermomixer F2.0 at 800-1500 rpm, and at either 50° C. for the DUOLITE™ A568, Lifetech™ ECR1640, LifeTech™ ECR1504, Lifetech™ ECR8309M, Chromolite™ D6154, and EziG™ Opal composition-carrier combinations 55° C. for the DUOLITE™ A561, DUOLITE™ PWA7, AmberLite™ FPA54 composition-carrier combinations. The reaction continued for 15-18 hours. The resulting product was developed on a HiPlex H Ligand Exchange column (Agilent) using an Agilent 1100 series HPLC system with in-line refractive index detector (0.6 mL/min with 5 mM H₂SO₄ mobile phase at 65° C.). Tagatose concentration was determined by comparing sample peak areas to those of known tagatose standard solutions. Immobilized enzyme composition cascade activity rates (μmol of tagatose produced/min/mg of total enzyme) were calculated for each carrier. Cascade activity rates for each carrier-composition preparation are reported in Table 1 relative to the cascade activity rate of the Duolite A568 carrier-composition preparation. Remaining maltodextrin and tagatose were washed off each immobilized enzyme preparation by five washes, each consisting of at least three volume equivalents of reaction buffer to equilibrate the preparation for re-use. The immobilized enzyme preparations were re-used by adding 2× concentrated maltodextrin feed solution as before. Reaction rates were calculated with each subsequent use and plotted to ascertain the working half-life of the immobilized catalyst. The half-life was determined by measuring the cascade rate (μmol/min/mg) on Day 0 and subsequent days until less than half the activity was lost consistently (compared to Day 0). A designation of n/d in Table 3 indicates <10% of the half-life was found or the cascade activity was <50% of Duolite A568.

TABLE 3

| | | Enzyme | Cascade Activity | Half-life (% |
|---|---|---|---|---|
| | | Immobilization | Rate (% relative | relative to |
| | | Interaction | to DUOLITE ™ | DUOLITE ™ |
| Carrier Name | Material | with Carrier | A568) | A568) |
| DUOLITE ™ A568 | phenol- | Ionic | 100.0% | 100% |
| | formaldehyde | | | |
| DUOLITE ™ A561 | phenol- | Ionic | 41.3%* | n/d |
| | formaldehyde | | | |
| DUOLITE ™ PWA7 | phenol- | Ionic | 92.0%* | 100% |
| | formaldehyde | | | |
| AMBERLITE ™ FPA54 | phenol- | Ionic | 25.7%* | n/d |
| | formaldehyde | | | |
| Lifetech ™ ECR1640 | polystyrene | Ionic | 35.9% | n/d |
| LifeTech ™ ECR1 | polystyrene | Ionic | 2.2% | n/d |
| Lifetech ™ ECR8309M | polymethacrylate | Ionic | 77.5% | 10% |
| Chromolite ™ D6154 | polymethacrylate | Affinity | 59.4% | 10% |
| EziG ™ Opal | controlled pore | Affinity | 119.6% | 30% |
| | glass | | | |

*saccharide to hexose reaction performed at 55° C.

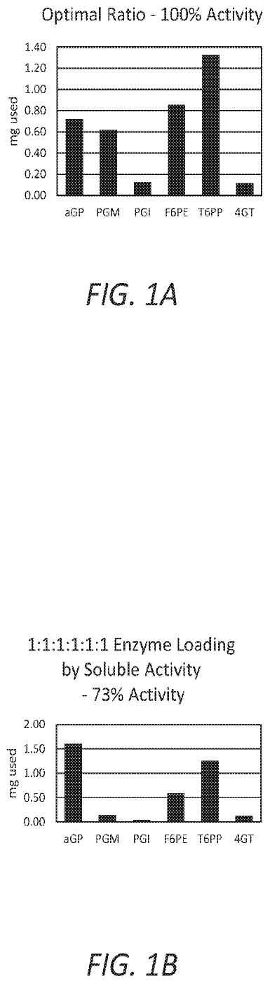

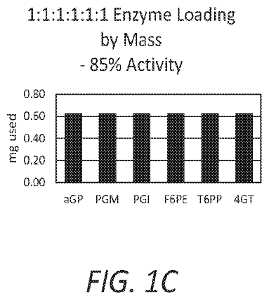

Example 2. Effect of enzyme ratio on cascade rate. To evaluate the effect of changing the ratios of various immobilized enzymes on tagatose production, the amounts of αGP, PGM, PGI, F6PE, T6PP, and 4-GT immobilized on DUOLITE™ A568 were varied relative to the DUOLITE™ A568-immobilized composition that was prepared using the enzyme ratios described in Example 1 and Table 1 (“the Example 1 immobilized composition”), in two, immobilized composition preparations. Their activity rates were evaluated against the performance of the Example 1 immobilized composition. In one of the varied-concentration immobilized compositions, the amounts of the enzymes were based on the observed rate of each enzyme relative to T6PP in solution, as shown in Table 4 (FIG. 1B), where an equal amount of enzyme activity (μmol/min) was added for each enzyme. The activity rate of this immobilized composition was 74% of the optimal ratio. The other varied-concentration immobilized composition contained equal amounts of each enzyme by weight (FIG. 1C). The activity rate of this immobilized composition was 85% of the optimal ratio. Reactions were carried out and conversion rates measured in μmol/min/mg total enzymes as described in Example 1.

TABLE 4

| | Enzyme activity as % of |
|---|---|
| Enzyme | T6PP activity |
| α glucan phosphorylase (αGP) | 78% |
| Phosphoglucomutase (PGM) | 861% |
| Phosphoglucoisomerase (PGI) | 2930% |
| fructose-6-phosphate epimerase (F6PE) | 215% |
| tagatose-6-phosphate phosphatase (T6PP) | 100% |
| 1,4-glucan transferase (4-GT) | 977% |

Example 3: Effect of distribution of enzymes on one or more carriers. The process of producing tagatose from maltodextrin using a DUOLITE™ A568-immobilized composition prepared in a single immobilization reaction as described in Example 1 (19% αGP, 17% PGM, 3% PGI, 23% F6PE, 35% T6PP, and 3% 4-GT), was compared to activity rates of processes performed using: (1) A mixture of a DUOLITE™ A568-immobilized composition of the core production enzymes (αGP, PGM, PGI, and 4GT) and a DUOLITE™ A568-immobilized composition of the tagatose production-specific enzymes (F6PE and T6PP); and (2) A mixture of 6, separate, immobilized enzyme preparations: DUOLITE™ A568 immobilized αGP, DUOLITE™ A568-immobilized PGM, DUOLITE™ A568-immobilized PGI, DUOLITE™ A568-immobilized 4GT, DUOLITE™ A568-immobilized F6PE and DUOLITE™ A568-immobilized T6PP. All the enzymes in (1) and (2) were present in the same ratios as in the referenced DUOLITE™ A568-immobilized composition, and all immobilizations were performed using a 5% w/w ratio (enzyme/carrier), and buffers/reaction conditions as described in Example 1. The results are shown in Table 5.

TABLE 5

| Co-immobilization configuration | Relative activity rate |
|---|---|
| αGP + PGM + PGI + 4GT + F6PE + | 100% |
| 4GT immobilized composition | |
| Combination of αGP + PGM + PGI + | 85.1% |
| 4GT immobilized composition and | |
| F6PE + T6PP immobilized composition | |
| Combination of separately immobilized | 27.2% |
| αGP + PGM + PGI + 4GT + F6PE + | |
| 4GT + F6PE + T6PP | |

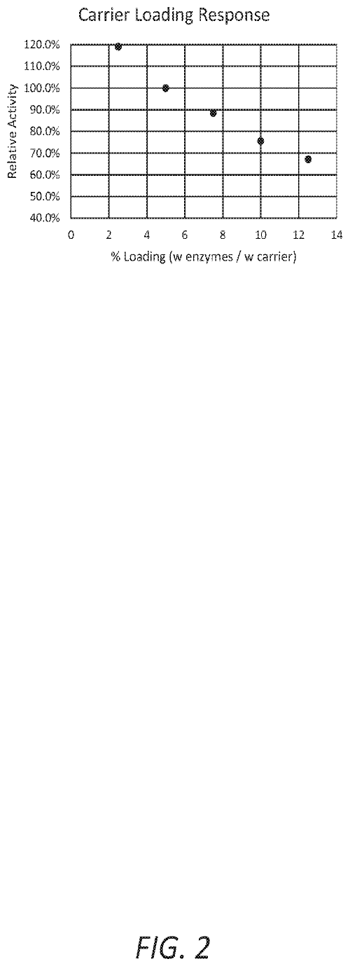

Example 4. Effect of enzyme loading. Enzymes at different loadings (g enzymes/g carrier ranging from 2.5% to 12.5% in 2.5% intervals) were immobilized on Duolite A568 as described in Example 1 and in the ratios listed in Table 2. Unlike Example 1, however, the immobilizations were allowed to continue for 16 hrs for each sample instead of 6 hrs to allow time for binding of enzymes to reach completion for the higher loadings. Soluble enzymes were washed off with five washes containing at least three sample volumes of reaction buffer pH 7.2 (25 mM Na₂PO₄, 4 mM Na₂SO₃, 2.5 mM MgS0₄, 0.25 mM MnCl₂). The samples were reacted with maltodextrin feed solution for 16 hours and conversion rates measured in μmol/min/mg total enzymes as in Example 1. The relative enzymatic cascade rates with respect to a 5% loaded sample are plotted as a function of loading in FIG. 2. The loading response together with the price of enzymes and support allows design of the most cost efficient catalyst.

Example 5. Allulose preparation using an immobilized enzyme composition. Enzyme compositions containing the following enzymes, αGP, PGM, PGI, psicose 6-phosphate 3-epimerase (P6PE), psicose 6-phosphate phosphatase (P6PP), and 4-GT in three different ratios among the enzymes were immoblized to DUOLITE™ A568 in respective immobilization reactions, as described in Table 6. A comparative analysis of the three immobilized composition preparations to produce allulose from maltodextrin was performed.

TABLE 6
Enzyme composition of allulose immobilization

| | | % w/w (enzyme/ | | |
|---|---|---|---|---|
| | | composition) | | |
| Enzyme | Uniprot ID | Prep 1 | Prep 2 | Prep 3 |
| α glucan phosphorylase | G8NCC0 | 20% | 20% | 20% |
| (αGP) | (SEQ. ID. NO. 1) | | | |
| Phosphoglucomutase | A0A0P6YKY9 | 16.5% | 16.5% | 16.5% |
| (PGM) | (SEQ. ID. NO. 2) | | | |
| Phosphoglucoisomerase | Q5SLL6 | 3% | 3% | 3% |
| (PGI) | (SEQ. ID. NO. 3) | | | |
| psicose 6-phosphate | A0A223HZ17 | 3% | 7% | 14% |
| 3-epimerase (P6PE) | (SEQ. ID. NO. 7) | | | |
| psicose 6-phosphate | A0A0E3NCH4 | 54% | 50% | 43% |
| phosphatase (P6PP) | (SEQ. ID. NO. 8) | | | |
| 1,4-glucan transferase | D7BF07 | 3% | 3% | 3% |
| (4-GT) | (SEQ. ID. NO. 6) | | | |

To prepare the immobilized cocktails, DUOLITE™ A568 was pretreated a 1% aqueous solution of glutaraldehyde (GA) for 2 hours at room temperature in an end-over-end rotator. The GA was removed by washing 5× with water and 2× conditioning washes with immobilization buffer (10 mM sodium phosphate buffer pH 7.2, 5 mM MgSO₄, and 80 μM CoCl₂). Enzyme solutions (Table 5) were then added to the GA-pretreated carrier after the final wash step (supernatant discarded). The enzyme solutions consisted of 5 g/L enzyme in reaction buffer (10 mM sodium phosphate buffer pH 7.2, 5 mM MgSO₄, 5 mM NaSO₃, and 80 μM CoCl₂). The enzyme plus carrier solutions were incubated at room temperature for 16 hours on an orbital shaker set at 800 rpm to immobilize the enzyme mixtures in the carrier. The total percent loading was 6.5% (mg of enzyme per mg of carrier). The supernatant was washed off with six washes consisting of reaction buffer to remove any remaining non-bound enzymes. The final supernatant was removed and 150 g/L maltodextrin, previously dissolved in reaction buffer was added. The maltodextrin—carrier mixture was shaken overnight (15-16 hours) in a 2.0 mL microfuge tube in an Eppendorf Thermomixer F2.0 at 800-1500 rpm and 55 ° C. The resulting product was developed on a SupelCogel Pb column (Sigma Aldrich) using an Agilent 1100 series HPLC system with in-line refractive index detector (0.6 mL/min with ultrapure water mobile phase at 80° C.). Allulose concentrations were determined by comparing sample peak areas to those of known allulose standard solutions, and enzymatic cascade specific activity rates calculated. The allulose production reaction was washed off each immobilization preparation with 4× washes of reaction buffer to equilibrate the immobilized preparation for re-use. The results of each sample composition's relative specific activities are shown below in Table 7.

TABLE 7
Relative activities of immobilized allulose production enzymes

| Immobilized Allulose | Relative Specific Activity |
|---|---|
| Compositions | (μmol/min/mg of enzyme) |
| Prep 1 | 100% |
| Prep 2 | 77.7% |
| Prep 3 | 51.1% |

Example 6. Allose preparation from maltodextrin using an immobilized enzyme composition. Allose will be produced from maltodextrin using an immobilized enzyme composition that includes αGP, PGM, PGI, 4GT and P6PE, A6PI, and A6PP.

Example 7. Fructose preparation from maltodextrin using an immobilized enzyme composition. Fructose will be produced from maltodextrin using an immobilized enzyme composition that includes αGP, PGM, PGI, 4GT, and F6PP.

Example 8. Mannose preparation from maltodextrin using an immobilized enzyme composition. Fructose will be produced from maltodextrin using an immobilized enzyme composition that includes αGP, PGM, PGI, 4GT, and M6PI or PGPMI and M6PP.

Example 9. Galactose preparation from maltodextrin using an immobilized enzyme composition. Galactose will be produced from maltodextrin using an immobilized enzyme composition that includes αGP, PGM, PGI, F6PE, 4GT, Gal6PI and Gal6P.

Example 10. Altrose preparation from maltodextrin using an immobilized enzyme composition. Altrose will be produced from maltodextrin using an immobilized enzyme composition that includes αGP, PGM, PGI, P6PE, Alt6PI, and Alt6PP.

Example 11. Talose preparation from maltodextrin using an immobilized enzyme composition. Talose will be produced from maltodextrin using an immobilized enzyme composition that includes αGP, PGM, PGI, F6PE, Tal6PI, and Tal6PP.

Example 12. Sorbose preparation from maltodextrin using an immobilized enzyme composition. Sorbose will be produced from maltodextrin using an immobilized enzyme composition that includes αGP, PGM, PGI, F6PE, S6PE, and S6PP.

Example 13. Gulose preparation from maltodextrin using an immobilized enzyme composition. Gulose will be produced from maltodextrin using an immobilized enzyme composition that includes αGP, PGM, PGI, F6PE, S6PE, Gul6PI, and Gul6PP.

Example 15. Idose preparation from maltodextrin using an immobilized enzyme composition. Idose will be produced from maltodextrin using an immobilized enzyme composition that includes αGP, PGM, PGI, F6PE, S6PE, 16PI, and I6PP.

Example 16. Inositol preparation from maltodextrin using an immobilized enzyme composition. Inositol will be produced from maltodextrin using an immobilized enzyme composition that includes αGP, PGM, 4GT, IPS, and IMP.

What is claimed:

1. An immobilized enzyme composition for the preparation of a hexose comprising at least two, at least three, at least four, at least five, at least six carriers, at least seven, or at least eight of the following enzymes immobilized to at least one carrier or a mixture of carriers:
a) a glucan phosphorylase (αGP), phosphoglucomutase (PGM), and optionally 1,4-glucan transferase (4-GT); and
b) an enzyme from within a combination of enzymes selected from:
(i) phosphoglucoisomerase (PGI), fructose-6-phosphate epimerase (F6PE), and tagatose-6-phosphate phosphatase (T6PP) to prepare tagatose;
(ii) phosphoglucoisomerase (PGI), piscose-6-phosphate epimerase (P6PE), and picose-6-phosphate phosphatase (P6PP) to prepare allulose;
(iii) phosphoglucoisomerase (PGI), P6PE, allose-6-phosphate isomerase (A6PI), and allose-6-phosphate phosphatase (A6PP) to prepare allose;
(iv) phosphoglucoisomerase (PGI), mannose-6-phosphate isomerase (M6PI) or phosphoglucose/phosphomannose isomerase (PGPMI), and mannose 6-phosphate phosphatase (M6PP) to prepare mannose;
(v) phosphoglucoisomerase (PGI), F6PE, galactose 6-phosphate isomerase (Gal6PI), and galactose 6-phosphate phosphatase (Gal6PP) to prepare galactose;
(vi) PGI and fructose 6-phosphate phosphatase (F6PP) to prepare fructose;
(vii) PGI, P6PE, altrose 6-phosphate isomerase (Alt6PI), and altrose 6-phosphate phosphatase (Alt6PP) to prepare altrose;
(viii) PGI, F6PE, talose 6-phosphate isomerase (Tal6PI), and talose 6-phosphate phosphatase (Tal6PP) to prepare talose;
(ix) PGI, F6PE, sorbose 6-phosphate epimerase (S6PE), and sorbose 6-phosphate phosphatase (S6PP) to prepare sorbose;
(x) PGI, F6PE, S6PE, gulose 6-phosphate isomerase (Gul6PI), and gulose 6-phosphate phosphatase (Gul6PP) to prepare gulose;
(xi) PGI, F6PE, S6PE, idose 6-phosphate isomerase (I6PI), and idose 6-phosphate phosphatase (I6PP) to prepare idose; and
(xii) inositol 3-phosphate synthase (IPS) and inositol monophosphatase (IMP) to prepare inositol.

2. The immobilized enzyme composition of claim 1, wherein the weight of each enzyme relative to the total weight of the enzymes (w/w) % ranges from 0.1% to 40%.

3. The immobilized enzyme composition of claim 1 or 2, comprising 10-30% (αGP); 0-10% (4GT); 10-30% (PGM); and when present 0.1-10% (PGI).

4. The immobilized enzyme composition of any one of claim 3, further comprising PGI, F6PE, and T6PP to prepare tagatose.

5. The immobilized enzyme composition of claim 4, wherein the weight of each enzyme relative to the total weight of the enzymes (w/w) % is: 10-30% (αGP); 0-10% (4GT); 10-30% (PGM); 0.1-10% (PGI); 15-35% (F6PE); and T6PP (25-45%), wherein the total weight of enzymes in the composition total 100 w/w % relative to the total weight of the enzymes.

6. The immobilized enzyme composition of claim 5, wherein
the αGP comprises the amino acid sequence, or fragment thereof, of SEQ ID NO. 1;
the 4-GT comprises the amino acid sequence, or fragment thereof, of SEQ ID NO. 6;
the PGM comprises the amino acid sequence, or fragment thereof, of SEQ ID NO. 2;
the PGI comprises the amino acid sequence, or fragment thereof, of SEQ ID NO. 3
the F6PE comprises the amino acid sequence, or fragment thereof, of SEQ ID NO. 4; and
the T6PP comprises the amino acid sequence, or fragment thereof, of SEQ ID NO. 5.

7. The immobilized enzyme composition of any one of claim 3, further comprising PGI, P6PE, and P6PP to prepare allulose.

8. The immobilized enzyme composition of claim 7, wherein the weight of each enzyme relative to the total weight of the enzymes (w/w) % is: 10-30% (αGP); 0-10% (4GT); 10-30% (PGM); 0.1-10% (PGI); 0.1-10% (P6PE); and (45-65%) P6PP, wherein the total weight of enzymes in the composition total 100 w/w % relative to the total weight of the enzymes.

9. The immobilized enzyme composition of claim 8, wherein:
the αGP comprises the amino acid sequence, or fragment thereof, of SEQ ID NO. 1;
the 4-GT comprises the amino acid sequence of SEQ ID NO. 6;
the PGM comprises the amino acid sequence of SEQ ID NO. 2;
the PGI comprises the amino acid sequence of SEQ ID NO. 3
the P6PE comprises the amino acid sequence of SEQ ID NO. 7; and
the P6PP comprises the amino acid sequence of SEQ ID NO. 8.

10. The immobilized enzyme composition of any one of claims 1-9, wherein the total weight of the enzymes relative to the weight of the carrier (w/w) % is from 2.5%-12.5%.

11. The immobilized enzyme composition of any one of claims 1-10, wherein the carrier is a weak base anion exchange resin.

12. The immobilized enzyme composition of claim 11, wherein the carrier comprises a phenol formaldehyde polycondensate.

13. The immobilized enzyme composition of claim 11 or 12, wherein the carrier comprises a tertiary amine functional group, wherein the composition is, optionally, DUOLITE™ A568.

14. The immobilized enzyme composition of claim 11 or 12, wherein the carrier comprises a secondary amine functional group, wherein the composition is, optionally, DUOLITE™ PWA7.

15. The immobilized enzyme composition of any one of claims 1-10, wherein the carrier comprises a His-tag affinity resin.

16. The immobilized enzyme composition of any one of claims 15, wherein the carrier comprises controlled pore glass (CPG) particles.

17. The immobilized enzyme composition of claim 15 or 16, wherein the carrier is functionalized by a chelated metal.

18. The immobilized enzyme composition of claim 18, wherein the chelated metal is iron or zinc or wherein the carrier is, optionally, EziG™ Opal.

19. An enzymatic process for preparing a hexose from a saccharide comprising the step of contacting a starch derivative with an immobilized enzyme composition of any one of claims 1-18 under suitable reaction conditions convert the starch derivative to the hexose.

20. The process of claim 19, wherein the process steps are conducted at a temperature ranging from about 40° C. to about 85° C., at a pH ranging from about 5.0 to about 8.0, and/or for about 0.5 hours to about 48 hours.

21. The process of claim 19 or 20, wherein the process steps are conducted in a single bioreactor, a plurality of bioreactors arranged in series, or a plurality of bioreactors arranged in parallel.

22. The process of any one of claims 19-21, wherein the process steps are conducted ATP-free, NAD(P)(H)-free, at a phosphate concentration from about 0.1 mM to about 150 mM, the phosphate is recycled within the enzymatic cascade reaction, and/or at least one step of the process involves a highly energetically favorable chemical reaction.
